(19) United States
(12) Patent Application Publication
(10) Pub. No.: US 2011/0162660 A1
(43) Pub. Date: Jul. 7, 2011

(54) Garment and Garment System

(76) Inventor: Claire Sanger, Kernersville, NC (US)

(21) Appl. No.: 12/986,110

(22) Filed: Jan. 6, 2011

Related U.S. Application Data
(60) Provisional application No. 61/292,609, filed on Jan. 6, 2010.
(60) Provisional application No. 61/363,502, filed on Jul. 12, 2010.

(51) Int. Cl. A61F 5/37; A41D 27/00

(52) U.S. Cl. 128/873; 2/255

(57) ABSTRACT

A garment system having a torso-covering garment with at least one internal sleeve for receiving a body positioner therein, and a body positioner removably insertable in the sleeve. The garment also has a retention member for releasably retaining the body positioner within the internal sleeve. When a person wearing the garment is in a supine position, the body positioner will position the person's upper torso in a predetermined orientation. The garment system may be used to prevent positional plagiocephaly in infants.

### CROSS REFERENCE TO RELATED APPLICATIONS

This application claims priority to U.S. Provisional Patent Application No. 61/292,609, filed on Jan. 6, 2010 and entitled Prevention of Roll-Over Garment System, and to U.S. Provisional Patent Application No. 61/363,502, filed on Jul. 12, 2010 and entitled Prevention of Roll-Over Garment System. The disclosures of both of the foregoing provisional patent applications are incorporated by reference herein.

### BACKGROUND

It is often desirable for infants, children and even adults to sleep in a particular position for a variety of reasons. For example, many people tend to snore louder and/or more frequently when sleeping on their back as compared to when they sleep on their side or stomach.

In the case of infants, beginning in 1992 the American Academy of Pediatrics (AAP) started the “Back to Sleep” campaign whereby the supine (on the back) sleep position, as opposed to sleeping on the stomach, was recommended for all infants. The purpose of this campaign was to decrease the incidence of Sudden Infant Death Syndrome (SIDS). While the campaign was successful in decreasing the occurrence of SIDS, it also resulted in an increase in positional plagiocephaly.

Positional plagiocephaly, also referred to deformational plagiocephaly or flat-head syndrome, is a condition in which a region of an infant's head becomes flattened. Positional plagiocephaly typically occurs in the occipital area (back) of the skull or on one side of the skull adjacent the occipital area. It is often caused by the infant sleeping with their head in the same position on the mattress or other supporting surface. Thus, although placing an infant in a supine sleeping position does decrease the incidence of SIDS, it can also cause positional plagiocephaly if the infant sleeps predominantly with the back of their head or one side of their head against the surface on which the infant lies. In particular, when an infant is placed in a supine position for sleep, the infant's head will often naturally rotate to one side or the other. Thus, although flattening of the skull may not necessarily occur in the occipital area of the skull, it will often occur on the side of the skull to which the infant's head naturally rotates when the infant is placed on their back for sleep.

The currently-accepted treatments for positional plagiocephaly include passive and active helmet therapy. The cost for the helmets can be $600-$3000. For families and caregivers, additional costs include time lost from work, travel and physician office visits.

In an effort to reduce the instance of positional plagiocephaly while not positioning infants on their stomach for sleep, parents and caregivers have resorted to using a variety of devices for preventing an infant from sleeping with their head in the same position. For example, some parents will place a folded or rolled towel under one side of the infant's back in order to cause the child's head to rotate in a direction away from the towel placement. A variety of other devices have also been marketed for similar purposes. However, towels and other devices used for this purpose can become displaced from their initial position. And such displacement can be dangerous, particularly if the displaced towel or device is pressed against the child's face or becomes wrapped around the child's neck.

While a variety of sleep positioning devices have been made and used, it is believed that no one prior to the inventor has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an infant wearing an exemplary garment system comprising an infant garment and inserted body positioner;

FIG. 2 depicts a front plan view of the garment system shown in FIG. 1;

FIG. 3 depicts a rear plan view of the garment system shown in FIG. 1, wherein a body positioner is shown being inserted into an internal sleeve provided in the garment;

FIG. 4 depicts a perspective view of the body positioner shown in FIG. 3;

FIG. 5 depicts a partially cut-away, rear plan view of the garment system shown in FIGS. 1-3, with the body positioner of FIG. 4 located in an internal sleeve provided in the garment;

FIG. 5A depicts an enlarged portion of FIG. 5 showing further details of a retention pouch, with the proximal end of a body positioner located therein;

FIG. 6 depicts a cross-sectional view of the garment system of FIG. 3;

FIG. 7 depicts an exploded rear plan view of the garment system of FIGS. 1-3;

FIG. 8 depicts a front plan view of an alternative embodiment of a garment system;

FIG. 9 depicts a rear plan view of the garment system shown in FIG. 8;

FIG. 10 depicts a rear plan view of the garment system shown in FIGS. 8-9, wherein a body positioner is shown being inserted into an internal sleeve provided in the garment;

FIG. 11 depicts an exploded rear plan view of the garment system of FIGS. 8-9;

FIG. 12 depicts a cross-sectional view of the lower portion of an internal sleeve of the garment system of FIG. 8;

FIG. 13 depicts a top plan view of a fabric panel for forming a retention pouch;

FIG. 14 depicts a perspective view of a retention pouch formed from the fabric panel of FIG. 13; and

FIG. 15 depicts a perspective view of an infant wearing yet another alternative embodiment of a garment system comprising an infant garment and inserted body positioner.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various examples of garments and garment systems having body positioners and/or features are depicted and described as being used for preventing positional plagiocephaly in an infant, the devices and features may be employed with other types of garments (e.g., for toddlers, children and/or adults). Accordingly, the drawings and descriptions should be regarded as illustrative in nature and not restrictive.

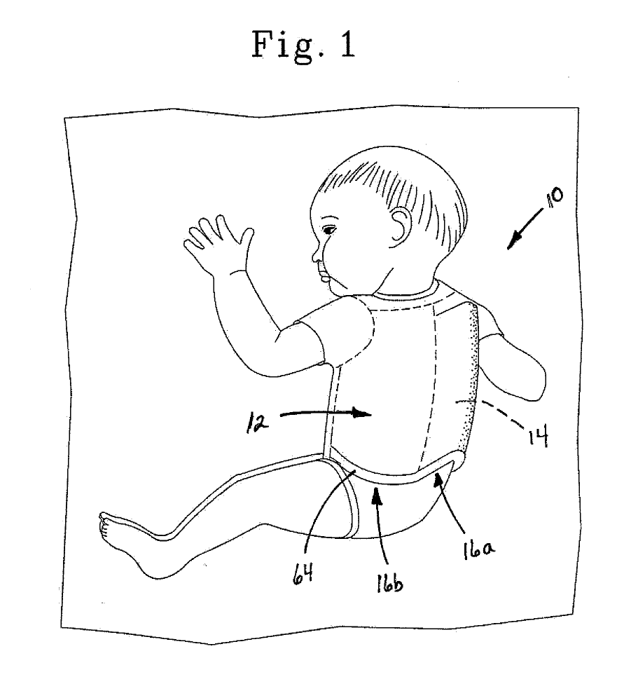

FIGS. 1-7 illustrate an exemplary garment system (10) and garment (12). As shown in FIG. 1, garment system (10) comprises a torso-covering garment (12) and a body positioner (or “bumper”) (14) removably located within an internal sleeve provided in the back of the garment. In the example shown, garment (12) is configured as an infant bodysuit having short sleeve members and very short leg members which encircle the upper portion of the legs adjacent the trunk of the infant. A lower portion of garment (12) extends over the crotch area of the infant, as further described herein. Similarly-configured, conventional infant bodysuits are sold under a variety of names, such as the Onesies® brand from Gerber Childrenswear. It will be understood, however, that garment (12) may be provided in any of a variety of configurations and sizes, including sleeveless configurations as well as configurations having long leg members which extend any desired distance along and around the infant's legs (including configurations having full length legs with integral booties, sometimes referred to as “sleepers” or “footed sleepers”).

As best seen in the rear plan view of FIG. 3, garment (12) includes a pair of internal sleeves (16a, 16b) located along either side of centerline (18) (also referred to herein as a “center stitch line”). Internal sleeves (16a, 16b) are not visible when garment (12) is worn by an infant, and garment (12) appears similar to a typical infant bodysuit—particularly when the body positioner is removed from garment (12). In addition, garment (12) is configured such that, the presence of the internal sleeves does not create excess bulk or otherwise detract from the comfort or aesthetics of garment (12). With or without the body positioner inserted into one of the sleeves, an infant will sleep comfortably in garment (12).

In the example shown, each internal sleeve (16a, 16b) generally extends from just above the uppermost outer end of leg cuffs (74a, 74b) to just below neck cuff (62). The outer side edge of each internal sleeve (16a, 16b) is defined by the sides (65a, 65b) of back panel (60) of garment (12), which generally coincide with the side seams along which the garment panels (described below) are stitched or otherwise joined to one another. When garment (12) is positioned on an infant, internal sleeves (16a, 16b) generally will be located on either side of the infant's spine, and extend from approximately the hips to the middle of the infant's shoulders (or higher) along either side of the spine.

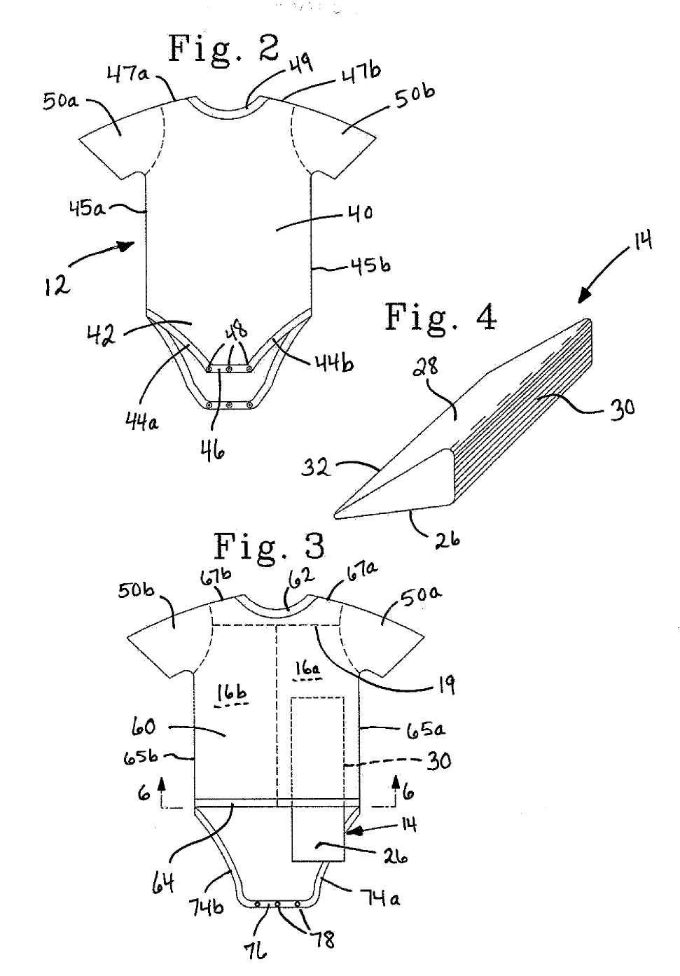

Body positioner (14) (as best seen in FIG. 4) comprises an elongate member sized and configured to be removably insertable into one of sleeves (16a, 16b). When inserted into one of sleeves (16a, 16b) of garment (12) worn by an infant, positioner (14) will be located against the infant's back lateral to the spine (as seen in FIG. 1). When the infant is laid on their back, body positioner (14) will elevate one side of the infant's back upwardly away from the surface on which the infant is laid, causing the infant's upper torso (above the hips) to be tilted with respect to the surface on which the infant lies. In other words, the side of the infant's torso adjacent positioner (14) will be elevated off of the supporting surface (e.g., a mattress), causing the infant's torso to be tilted generally about their spine.

Since an infant's neck muscles are typically weak, such tilting of the torso will also cause the infant's head to rotate in a direction away from the side of garment (12) wherein positioner (14) is placed. In the example shown in FIG. 1, body positioner (14) has been inserted into right sleeve (16a) of garment (12). When the infant of FIG. 1 is laid on his back, elevation of the right side of the infant's torso by positioner (14) will result in the infant's head rotating to the left. This rotation will ensure that the infant does not sleep with the back of his head flat against the supporting surface (e.g., a mattress).

While causing an infant's head to rotate to one side while the infant is sleeping in a supine position will help to prevent flattening of the occipital region of the skull, garment system (10) is configured so that this head rotation may be periodically reversed to the opposite side. Such periodic reversal of head rotation will help prevent flattening of the skull on either side caused by the infant sleeping with their head rotated in the same direction. Since garment (12) has two sleeves (16a, 16b) located on opposite sides of centerline (18), by alternating the placement of positioner (14) within sleeves (16a, 16b) the rotation of the infant's head when the infant is supine will be alternated as well. Thus, when positioner (14) is inserted into left sleeve (16b), the elevation of the left side of the infant's torso by positioner (14) will result in the infant's head rotating to the right. In this manner, garment system (12) may be used to prevent positional plagiocephaly by periodically moving body positioner (14) from one sleeve to the other, or removing it entirely. By way of example, body positioner placement may be changed (inserted into one of the sleeves, moved from one sleeve to the other, or removed from garment (10)) every two hours while the infant is awake, and before every sleep cycle in order to repeatedly alter the position of the infant's head.

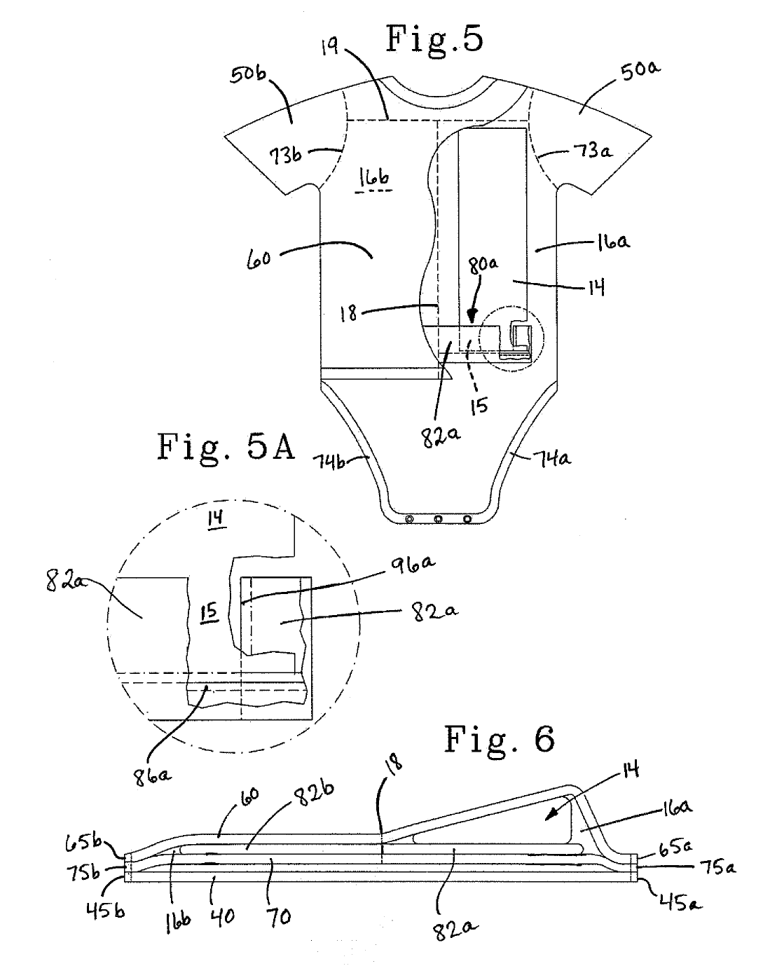

Body positioner (14) may be provided in any of a variety of shapes and sizes. In some embodiments, including the example shown in FIGS. 1-7, positioner (14) is sized so as to fit snugly within sleeves (16a,16b), while also being easy to remove therefrom. Thus, as best seen in the partial cut-away view of FIG. 5, positioner (14) is slightly shorter in length, and narrower than sleeve (16a, 16b). When inserted into a sleeve (16a,16b) of garment (12) on an infant, positioner (14) extends from approximately the hips to approximately the middle of the infant's shoulder (just below the neckline of garment (12)). As further described herein, sleeves (16a, 16b) are narrower at their upper ends (i.e., adjacent the shoulders). This allows the distal (upper) end of body positioner (14) to fit more tightly in the upper end of sleeves (16a, 16b), while providing a less snug fit in lower regions of sleeve (16a, 16b). One or more retention members (further described herein) may also be provided for releasably retaining the body positioner (14) within sleeve (16a, 16b). Of course it will be understood that positioner (14) may be provided in any of a variety of sizes, depending in part on the configuration of garment (12) and the sleeves (16a, 16b) provided therein.

Body positioner (14) also may be provided in a variety of shapes, such as the elongate wedge-shaped member having a triangular cross-section shown in FIG. 4. Elongate members of various other cross-sectional shapes may also be employed, such as round or oval cross-sections. In other embodiments, the body positioner is an elongate wedge-shaped member wherein the top surface of the wedge-shaped member is slightly concave or otherwise shaped to more closely match the shape of an infant's back. In the example shown, positioner (14) includes a bottom surface (26), a top surface (28), an outer sidewall (30), and an inner sidewall (32). Positioner (14) is thickest at outer sidewall (30), and thinnest at inner sidewall (32). In one embodiment, bottom surface (26) and top surface (28) intersect a cross-sectional plane through positioner (14) at an angle of 15 to 32 degrees. In other embodiments, this angle may be increased (e.g., up to 45 degrees, particularly if the garment system (12) is configured to be worn by larger individuals. Such increased angle will provide additional support without necessarily increasing the rigidity (or firmness) of the positioner (14).

As best seen in FIG. 3, positioner (14) may be inserted into sleeve (16a, 16b) such that bottom surface (26) faces rearwardly (i.e., away from the back of an infant wearing garment (12)), with outer sidewall (30) adjacent side edge (65a, 65b) of back panel (60) of garment (12). The thinnest cross-sectional portion of positioner (14), namely inner sidewall (32), is located adjacent centerline (18) of garment (12). In this manner, when an infant wearing garment (12) with positioner (14) inserted into right sleeve (16a) is laid in a supine position on a supporting surface (e.g., a mattress), top surface (28) of positioner (14) will elevate the right side of the infant's torso upwardly away from the supporting surface. Such tilting of the torso will also result in the infant's head rotating to the left.

Although FIGS. 3 and 5 show positioner (14) inserted into right sleeve (16a), positioner (14) may be similarly inserted into left sleeve (16b) simply by rotating positioner (14) 180 degrees so that outer sidewall (30) is adjacent left side edge (65b) of rear panel (60) of garment (12). When an infant wearing garment (12) with positioner (14) inserted into left sleeve (16b) is placed in a supine position on a supporting surface (e.g., a mattress), top surface (28) of positioner (14) will elevate the left side of the infant's torso away from the supporting surface. Such tilting of the torso will also result in the infant's head rotating to the right.

As discussed further herein, in the example shown in FIGS. 1-7 positioner (14) may be inserted into sleeve (16a, 16b) such that inner sidewall (32) is nearer to centerline (18) than outer sidewall (30) is to the side edges (65a, 65b) of back panel (60) of garment (12). Such positioning not only allows positioner (14) to extend above the midpoint of the sleeves, just below the neckline, but also facilitates insertion of positioner (14) into sleeve (16a, 16b). In addition, the higher the distal end of positioner (14) is with respect to the neck, the more difficult it will be for the infant to rotate their head in the direction of positioner (14).

Positioner (14) may be made from any of a variety of materials which have sufficient strength and rigidity to elevate one side of the infant's torso off of the supporting surface on which the infant is laid. In some embodiments, the material is resilient such that portions of body positioner (14) will be compressed by the weight of the infant. In this manner, the upper surface (28) of positioner (14) will, to some extent conform to the shape of the infant's back, thus altering pressure points and reducing the likelihood of pressure sores. A resilient positioner (14) will also be less restrictive of the infant's movement as compared to a rigid or firm positioner.

By way of example, positioner (14) may be made from a polymeric foam. In one example, positioner (14) comprises reticulated or open-celled foam, such as reticulated polyurethane foam. Such foams are often very washable, breathable and dry quickly. Breathability of the foam or other material used to fabricate positioner (14) will provide greater comfort to the infant, and limit perspiration between the infant's back and positioner (14). In addition to reticulated or open cell foam or other polymeric materials, various outer coverings may be provided on positioner (14). Such coverings may be employed for any of a variety of reasons such as improving retention of the positioner (14) within the sleeve (16a, 16b), facilitating removal of positioner (14) from the sleeve (16a, 16b), improving durability and/or washability, etc.

In one example, body positioner (14) comprises a reticulated polyether polyurethane foam having a reactive antimicrobial agent which will not leach from the foam if positioner (14) is washed or soiled. The foam has 20 to 30 pores per inch (PPI), a density of 1.7 pounds per cubic inch, and in indentation force deflection (IFD) of 30 to 60 pounds.

Internal sleeves for receiving a body positioner therein may be provided in a garment in a variety of ways. In some embodiments (such as that shown in FIGS. 1-7 herein), the garment is configured such that the sleeves are not visible. In addition, in these embodiments the positioner is not visible when inserted into a sleeve. While the positioner in these embodiments will manifest its presence by an unobtrusive protrusion visible from the rear of the garment, it will not detract from the overall aesthetics of the garment (including any surface patterns or other ornamentation provided on the garment). In some views herein, stitching definining the positioner-receiving sleeves is depicted—particularly in rear views of the various garments. It will be understood, however, that the appearance of the stitching is exaggerated for purposes of clarity. In physical garments produced in accordance with the embodiments described herein, such stitching will typically be barely visible (like other stitching on garments). It should also be pointed out that the thickness of the various panels shown in the figures, particularly the cross-sectional views, have been greatly exaggerated in order to facilitate an understanding of the described embodiments.

Also in the depicted embodiments, even though the sleeves for receiving positioner (14) are located within the garment rather than being attached to the rear layer or face of the garment, the body positioner (14) may be inserted and removed from an internal sleeve while the garment is being worn by an infant. Furthermore, since the exemplary embodiments have a sleeve located to either side of the centerline of the garment, the positioner may be inserted or removed while an infant wearing the garment is in a supine position. For example, access to a sleeve for insertion or removal of the positioner when the infant is supine may be obtained merely by lifting one side of the infant. This is further facilitated, in some embodiments, by locating the sleeve access openings below or adjacent the lower ends of their respective sleeves. In the depicted embodiments it is not necessary to pick the infant completely off of a supporting surface (e.g., a mattress) on which the infant lay. This feature of some embodiments facilitates the alternating placement of the body positioner in the right and left sleeves, since the positioner may be removed from one sleeve and then inserted into the other sleeve without waking or overly disturbing a sleeping infant.

In the embodiment shown in FIGS. 1-7, garment (12) generally includes a front panel (40), sleeve members (50a, 50b), and back panel (60). Front panel (40) and back panel (60) are stitched or otherwise fastened to one another along their respective sides (45a, 45b)(65a, 65b). Similarly, sleeve members (50a, 50b) are stitched or otherwise fastened to both front panel (40) and back panel (60) in the usual fashion. In this manner, front panel (40) and back panel (60) together form garment (12) which is configured to be worn over the torso (i.e., from the hips to, and over, the shoulders). As mentioned previously, sleeve members (50a, 50b) may be omitted to provide a sleeveless configuration, or may be extended so as to provide a long sleeve configuration for garment (12). It will also be understood that front panel (40), back panel (60), and sleeve members (50a, 50b) may be provided by more than one section of material. For example, gussets may be provided in the armpit area. Or, one or more of the panels may comprise multiple sections of fabric joined to one another, such as by stitching. Furthermore, front panel (40) and back panel (60) may even be formed from a single piece of fabric such that, for example, the front and back panels are stitched to one another along only one side edge of garment (12).

In the embodiments shown and described herein, garment (12) is configured to be worn by an infant. To prevent the garment from riding up on the infant (which may cause serious harm to the infant), infant garments which lack full leg portions (sometimes referred to as infant bodysuits) include portions which extend below the waist and encircle the upper portions of the legs adjacent the trunk as well as over the crotch and buttocks areas.

Front panel (40) includes a lower portion (42) which, when garment (12) is placed on an infant, extends over the front crotch area. Lower portion (42) terminates in right and left leg cuffs (44a, 44b), and center cuff (46) located therebetween. Center cuff (46) includes one or more snap fasteners (48) which are used to engage mating snap fasteners provided on the portion of garment (12) which extends over the buttocks area of an infant wearing the garment. Such a selectively closeable crotch portion allows diaper changing without removing the entire garment. It will be understood that various other types of fastener other than snap fasteners may be provided, such as buttons, a zipper, a hook and loop fastening system, or other arrangements known to those skilled in the art. In addition, the crotch portion of garment (12) may be stitched closed rather than providing a selectively closeable arrangement. A neck cuff (49) may be provided at the upper end of front panel (40), as shown.

Back panel (60) is shown in the rear plan view of FIG. 3, and generally includes a neck cuff (62) at its upper end. Back panel (60) is also shaped similarly to front panel (40) from the waist region up. In this fashion, back panel (60) and front panel (40) are stitched to one another along their respective sides (65a, 65b)(45a, 45b) and at their respective upper ends (67a, 67b)(47a, 47b), and both are stitched to sleeve members (50a, 50b), thus forming torso-covering garment (12). At its lower end, back panel (60) terminates in a lower cuff (64) which extends laterally across garment (12), just above the uppermost outer ends of leg cuffs (44a, 44b) of front panel (40). Lower cuff (64) is not attached to front panel (40). As further described below, body positioner (14) may be inserted into sleeve (16a, 16b) by sliding positioner (14) into the interior of the garment under lower cuff (64) (as shown in FIG. 3).

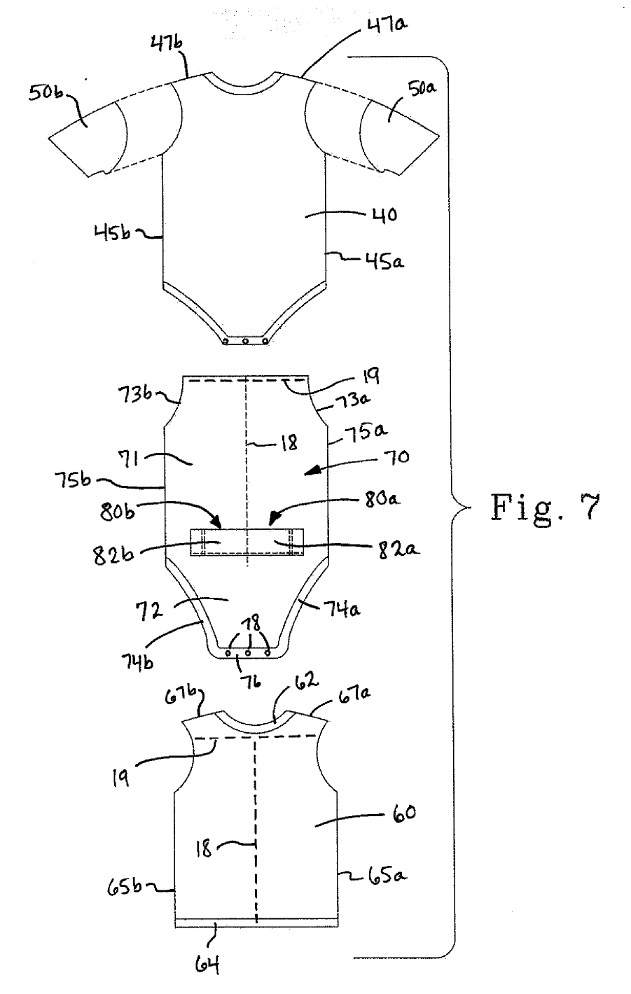

In order to provide internal sleeves (16a, 16b), an inner panel (70) is located between front panel (40) and back panel (60). Inner panel (70) is stitched or otherwise fastened to back panel (60) such that sleeves (16a, 16b) are provided between facing regions of back panel (60) and inner panel (70). In the embodiment shown in FIGS. 1-7, and as best seen in FIG. 7, inner panel (70) generally comprises an upper portion (71) and a lower portion (72). Upper portion (71) is sized and configured such that inner panel (70) is stitched between front panel (40) and back panel (60) along sides (75a, 75b) (as shown in the cross-sectional view of FIG. 6). Similarly, upper curved edges (73a, 73b) are stitched between front panel (40) and back panel (60) where sleeve members (50a, 50b) are joined to the front and back panels. It will be understood, however, that inner panel (70) may be configured in a variety of alternative ways.

Lower portion (72) of inner panel (70) extends downwardly away from sides (75a, 75b) and is not stitched to either front panel (40) or back panel (60). When garment (12) is placed on an infant, lower portion (72) extends over the buttocks area. Lower portion (72) terminates in right and left leg cuffs (74a, 74b), and center cuff (76) located therebetween. Center cuff (76) includes one or more snap fasteners (78) which are used to matingly engage snap fasteners (48) on lower portion (42) of front panel (40) which extends over the front crotch area of an infant wearing the garment. In this manner, when fasteners (48) and (78) are engaged with one another, lower portions (42, 72) on front panel (40) and inner panel (70), respectively, will extend over and around the crotch and buttocks areas of the infant. Such an arrangement will maintain garment (12) in place on the infant and prevent the garment (12) from riding up in the direction of the infant's head.

Inner panel (70) is also stitched to back panel (60) along center stitch line (18) and upper stitch line (19). Upper stitch line (19) extends across the entire width of inner panel (70), adjacent the upper edge of inner panel (70). On back panel (60), upper stitch line (19) extends across the entire width of back panel (60), adjacent neck cuff (62). On inner panel (70), center stitch line (18) extends from upper stitch line (19) down the center of inner panel (70) to the lower end of upper portion (71). On back panel (60), center stitch line (18) extends from upper stitch line (19) down the center of back panel (60) through lower cuff (64). Such stitching of inner panel (70) to back panel (60) results in the formation of sleeves (16a, 16b) which extend from just above lower cuff (64) to upper stitch line (19), between center stitch line (18) and sides (65a/75a, 65b/75b) of inner panel (70) and back panel (60).

As shown in FIG. 3, body positioner (14) is inserted into sleeve (16a, 16b) by sliding the distal (upper) end of positioner (14) beneath lower cuff (64), and thereafter urging positioner (14) upwardly until the distal end of positioner (14) is located adjacent upper stitch line (19), between center stitch line (18) and upper curved edge (73a, 73b) of inner panel (70). Thus, openings for gaining access to sleeves (16a, 16b) are provided between lower cuff (64) of back panel (60) and the rearwardly facing (i.e., outer) surface of inner panel (70), with an opening provided on either side of centerline (18).

Upper curved edges (73a, 73b) are stitched between the front and back panels and the sleeve members (50a, 50b), thus making internal sleeves (16a, 16b) somewhat narrower at the upper end thereof. Positioner (14) is configured so that its distal end may be snugly positioned between upper curved edge (73a, 73b) and center stitch line (19). In this manner, the snug fit of the distal end of positioner (14) will help retain positioner (14) within sleeve (16a, 16b). In addition, although the width of back panel (60) is such that back panel (60) loosely overlies inner panel (70) to allow positioner (14) to be easily inserted into sleeve (16a, 16b) between back panel (60) and inner panel (70), back panel (60) may nevertheless exert some compressive force on positioner (14), further helping retain posititioner (14) within sleeve (16a, 16b). The material of positioner (14) and/or a protective covering on positioner (14) may also be selected to provide increased friction between the fabric of garment (12) and positioner (14) to further help retain positioner 14 within sleeve (16a, 16b).

On the other hand, it may be desirable to not only retain positioner (14) within sleeve (16a, 16b), but also facilitate the insertion and removal of positioner (14). Thus, while providing a tighter fit of positioner (14) within sleeve (16a, 16b) (e.g., by providing a closer fit between back panel (60) and inner panel (70)) will help to retain positioner (14) within the sleeve, it will be more difficult to insert and remove the positioner. For this reason, garment (12) shown in FIGS. 1-7 further includes retention members for retaining the positioner (14) within sleeve (16a, 16b). Such retention members may have any of a variety of forms, such as internal elastic straps or bands which help to retain positioner (14) within a sleeve (16a, 16b).

In the example shown, the retention members comprise retention pouches (80a, 80b) located at the bottom of each sleeve (16a, 16b). Each retention pouch (80a, 80b) is provided by one or more fabric panels stitched to either the inner surface of back panel (60) adjacent lower cuff (64) or the rearwardly facing surface of inner panel (70) at the same location. In the example shown, retention pouches (80a, 80b) are provided by gusseted panels (82a, 82b) which are attached along their sides and bottom edge to inner panel (70) adjacent the lower end of upper portion (71) of inner panel (70), along either side of center stitch line (18).

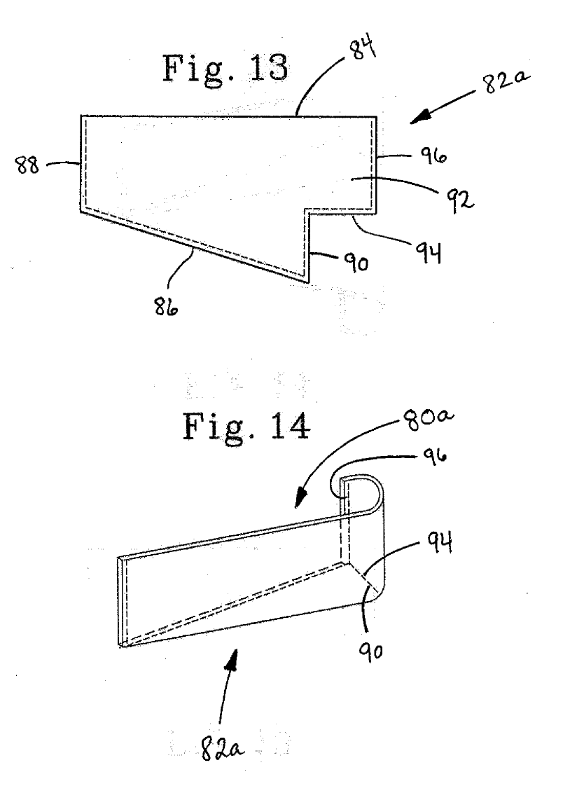

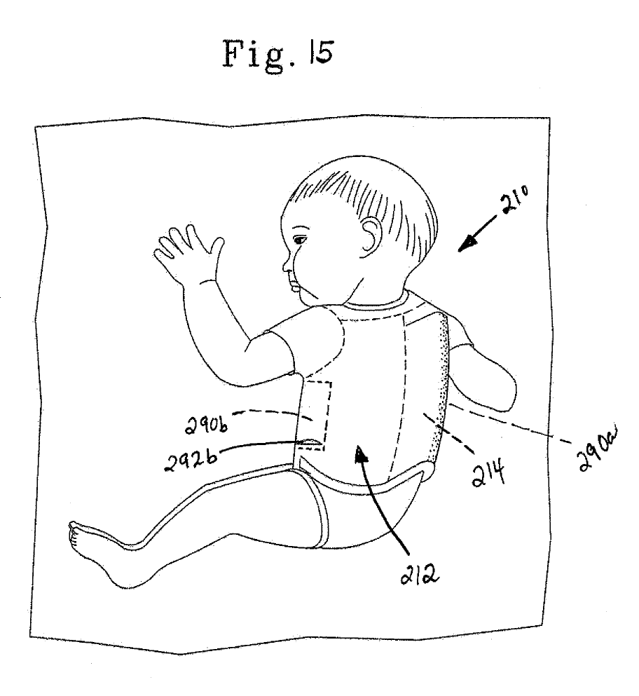

One exemplary gusseted fabric panel (82a) is shown in FIGS. 13 and 14. Panel (82a) has a straight upper edge (84), a lower edge (86) which extends at an angle to upper edge (84), inner side edge (88) extending between upper edge (84) and lower edge (86), and right side edge (90) which extends upwardly away from lower edge (86) to gusset portion (92). Gusset portion (92) has a lower edge (94) and a right side edge (96). In order to form retention pouch (80a), right side edge (90) is stitched to lower edge (94) of gusset portion (92). Left side edge (88) is stitched to inner panel (70) at or adjacent to center stitch line (18) (i.e., left side edge (88) is stitched between back panel (60) and inner panel (70) at center stitch line (18)). Bottom edge (86) is stitched to inner panel (70) just above where lower cuff (64) of back panel (60) overlays inner panel (70). And right side edge (96) of gusset portion (92) is stitched to inner panel (70) along a vertical line parallel to, and spaced inwardly from, side edge (75a) of inner panel (70). Since the upper edge (84) of panel (82a) is not stitched to the inner panel (70), pouch (80a, 80b) is formed at the lower end of each sleeve (16a, 16b), just above lower cuff (64) of back panel (60). In this manner, and as shown in the partial cut-away view of FIGS. 5 and 5A, after positioner (14) has been slid into sleeve (16a, 16b), the proximal (lower) end (15) of positioner (14) may be inserted into retention pouch (80a, 80b). This may be accomplished by reaching up into sleeve (16a, 16b) beneath lower cuff (64) of back panel (60), grabbing upper edge (84a, 84b) of panel (82a, 82b), and pulling downward. The pouch (80a, 80b) may thus be pulled around and then over the proximal end of body positioner (14).

As also seen in FIG. 14, pouch (80a, 80b) has a tapered width which corresponds to the cross-sectional shape of the proximal end of positioner (14). By providing corresponding sizes and shapes in pouch (80a, 80b) and the proximal end (15) of positioner (14), particular if the dimensions of pouch (80a, 80b) are slightly smaller than that of proximal end (15), the elasticity of the fabric used for panel (82a, 82b) will allow retention pouch (80a, 80b) to compressively hold the proximal end (15) of positioner (14). By providing such an arrangement, garment (12) may be configured such that the portion of positioner (14) located between its distal and proximal ends fits more loosely within sleeve (16a, 16b). In this manner, body positioner (14) is easier to insert and remove from sleeve (16a, 16b), while still preventing positioner (14) from becoming inadvertently displaced or dislodged.

The fabric layer(s) used to form retention pouch (80a, 80b) may have any of a variety of other shapes and sizes. For example, a rectangular piece of fabric may simply be stitched to inner panel (70) along the side and bottom edges of the fabric to form retention pouch (80a, 80b). Any of a variety of other shapes and configurations may be used, depending in part on the cross-sectional shape of positioner (14). Also, fabric panels (82a, 82b) may be stitched to the inner surface of back panel (60), rather than inner panel (70). In this alternative arrangement, the retention pouch will hold the proximal end of the postioner (14) against a lower end region of back panel (60) rather than against inner panel (70).

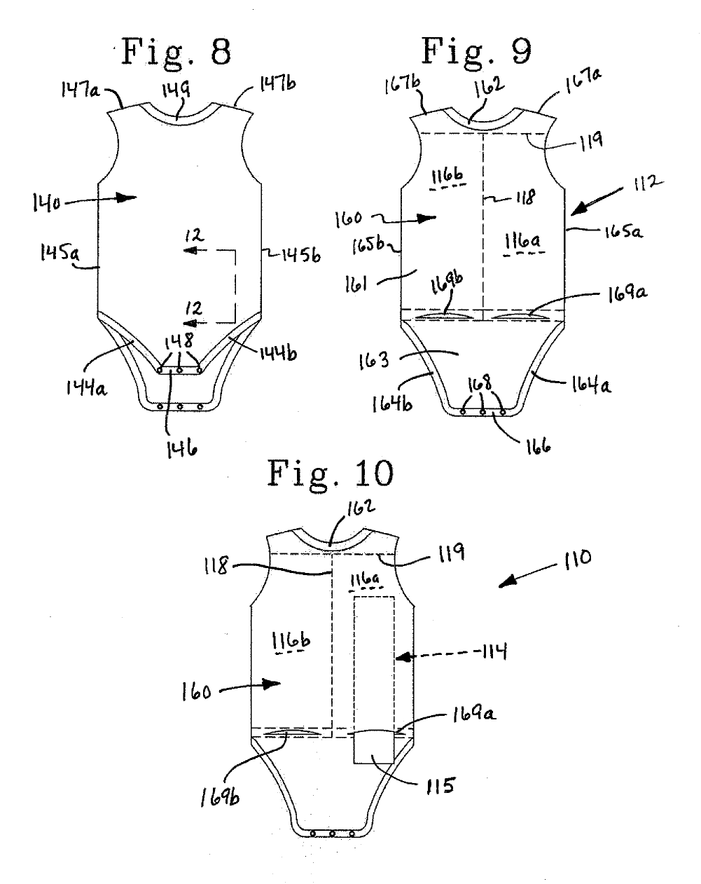

FIGS. 8-12 depict an alternative garment system (110) suitable for use, for example, in preventing positional plagiocephaly in an infant. Garment system (110) is similar to garment system (10) described previously, and comprises a garment (112) and a body positioner (114) which is inserted into an internal sleeve (116a, 116b) located within garmet (112). Positioner (114) is configured similarly to that described previously with respect to positioner (14).

Like garment (12), garment (112) shown in FIGS. 8-12 is depicted in the configuration of an infant bodysuit. In this instance, however, garment (112) is sleeveless. Garment (112) is also similar to garment (12) described previously in that it includes a front panel (140) and a back panel (160), wherein the front and back panels are joined together (e.g., stitched) along their respective sides (145a, 145b)(165a, 165b), and along their respective upper ends (147a, 147b)(167a, 167b). When attached in this fashion, front panel (140) and back panel (160) form a garment structure which may be placed about an infant's torso.

Like front panel (40), front panel (140) also includes a lower portion (142) which, when garment (112) is placed on an infant, extends over the front crotch area. Lower portion (142) also terminates in right and left leg cuffs (144a, 144b), and center cuff (146) located therebetween. Center cuff (146) includes one or more snap fasteners (148) which matingly engage with snap fasteners provided on lower portion (163) of back panel (160) (as further described herein). As mentioned previously with respect to garment (12), the crotch portion of garment (112) may be stitched closed rather than providing a selectively closable arrangement as shown.

As seen in the rear plan view of FIG. 9, back panel (160) generally includes a neck cuff (162) at its upper end which, together with neck panel (149) on front panel (140), encircle an infant's neck when garment (112) is positioned on the infant. Back panel (160) is also shaped similarly to front panel (140) and therefore includes an upper portion (161) and a lower portion (163) extending downwardly therefrom. Thus, unlike back portion (60) of previously described garment (12), back panel (160) includes lower portion (163). When garment (112) is placed on an infant, lower portion (163) extends over the buttocks area. Lower portion (163) terminates in right and left leg cuffs (164a, 164b) and center cuff (166) located therebetween. Center cuff (166) includes one or more snap fasteners (168) which are used to matingly engage snap fasteners (148) on lower portion (142) of front panel (140) which extends over the front crotch area of an infant wearing the garment (112). In this manner, when these two sets of fasteners (148, 168) are engaged with one another, lower portions (142, 163) of front panel (140) and back panel (160), respectively, will extend over and around the crotch and buttocks area of the infant. Such an arrangement will maintain garment (112) in place on the infant and prevent the garment (112) from riding up in the direction of the infant's head. It will therefore be seen that lower portion (163) of back panel (160) functions similarly to lower portion (172) of inner panel (170) of garment (12) described previously.

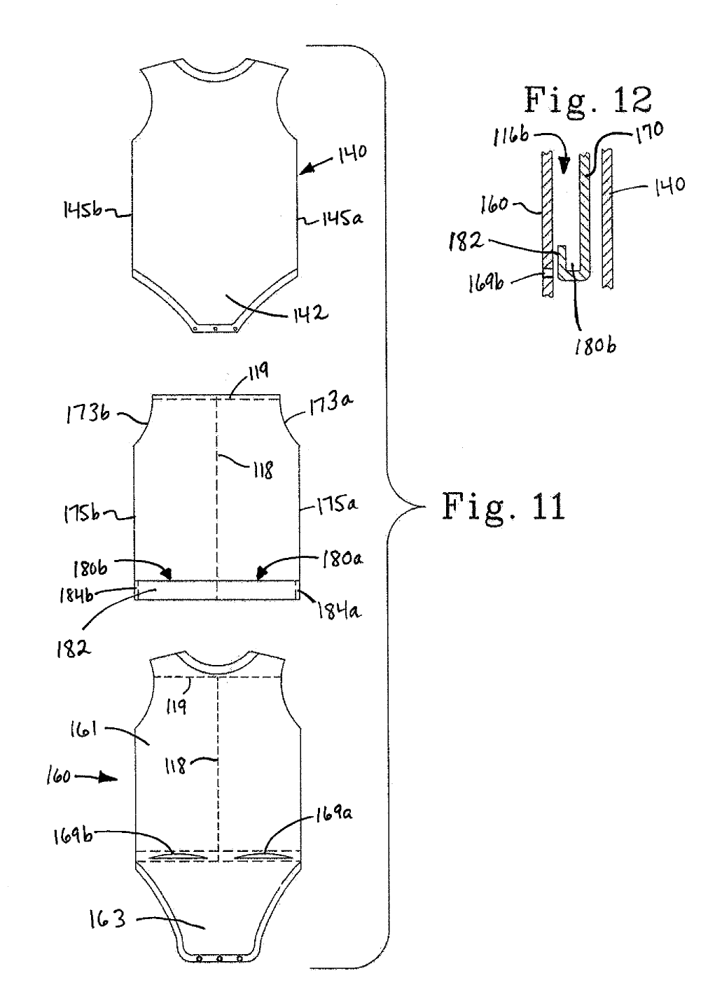

In order to provide internal sleeves (116a, 116b) in garment (112), an inner panel (170) is provided between front panel (140) and back panel (160). As best seen in the rear exploded view of FIG. 11, inner panel (170) is shaped similar to front panel (60) of the previously-described embodiment. Inner panel (170) is stitched or otherwise fastened to back panel (160) such that sleeves (116a, 116b) are provided between facing regions of back panel (160) and inner panel (170). Inner panel (170) is also sized and configured such that sides (175a, 175b) of inner panel (170) are stitched between sides (45a, 45b)(65a, 65b) of front panel (140) and back panel (160), respectively. Similarly, upper curved edges (173a, 173b) of inner panel (170) are stitched between similarly-shaped upper curved edges provided on front panel (140) and back panel (160).

Inner panel (170) is also stitched to back panel (160) along center stitch line (118), upper stitch line (119) and lower stitch line (121). Upper stitch line (119) extends across the entire width of inner panel (170) just below the uppermost edge of inner panel (170). On back panel (160) upper stitch line (119) extends across the entire width of back panel (160) just below neck cuff (162). Center stitch line (118) extends down inner panel (170) from upper stitch line (119), down the center of inner panel (170) to bottom stitch line (121). On back panel (160), center stitch line (118) extends from upper stitch line (119) down the center of back panel (162) just above lower cuffs (164a, 164b) on back panel (160) (i.e., to lower stitch line (121)).

If desired, the lower edge of inner panel (170) may be stitched to back panel (160), below openings (169a, 169b), thus providing sleeves (116a, 116b) between inner panel (170) and back panel (160) with one sleeve positioned on either side of center stitch line (118).

In the embodiment shown, however, the lowermost edge of inner panel (170) is not stitched or otherwise affixed to back panel (160). Instead, retention pouches (180a, 180b) are provided on inner panel (170) at the bottom of sleeves (116a, 116b). Retention pouches (180a, 180b) are located at the lowermost end of inner panel (170), and are configured to receive the proximal (lower) end (115) of positioner (114). Retention pouches (180a, 180b) may be provided in a variety of ways. In the exemplary embodiment shown, the lower end portion (182) of back panel (170) is folded up as shown in FIGS. 11 and 12. Lower end portion (182) is stitched between back panel (160) and inner panel (170) along outer side edges (184a, 184b), thus forming retention pouches (180a, 180b).

In order to allow positioner (114) to be inserted into sleeves (116a, 116b), openings (169a, 169b) are provided in back panel (160). Openings (169a, 169b) extend through back panel (160), and are located to provide access to sleeves (116a, 116b). In the embodiment shown, openings (169a, 169b) comprise slits which are slightly wider than the width of positioner (114) so as to allow positioner (114) to be inserted therethrough into sleeve (116a, 116b). In this fashion, openings (169a, 169b) will have the appearance of a large button hole. Any of a variety of other shapes may be used for openings (169a, 169b), depending in part on the shape of positioner (114).

Openings (169a, 169b) are generally located near the bottom of upper portion (161) of back panel (160) in order to facilitate insertion of positioner (114). In the particular embodiment shown, openings (169a, 169b) are located adjacent lower end portion (182) of inner panel (170) so that openings (169a, 169b) are positoned at or near the bottom of retention pouches (180a, 180b). Since the fabric used for back panel (160) will generally be capable of being stretched to some extent, when positioner (114) is inserted into one of openings (169a, 169b), the bottom edge of the opening may be pulled downwardly in order to insert the proximal end of positioner (114) into the retention pouch (180a, 180b). When retention pouches (180a, 180b) are not provided, the proximal end (126) of positioner (114) is simply positioned within the lower portion of the sleeve (116a, 116b) located below the opening (169a, 169b). With retention pouches (180a, 180b), once positioner (114) has been slid completely into the sleeve (116a, 116b), the user simply pulls downwardly on opening (169a, 169b) and lower end portion (182) of inner panel (170) so that the retention pouch (180a, 180b) is pulled over the proximal end (115) of positioner (114) so as to retain proximal end (115) within the pouch (180a, 180b).

It should also be pointed out that openings (169a, 169b) may be located elsewhere, such as just below neck cuff (162) at (or near) the upper end of sleeves (116a, 116b).

Since the fabric of back panel (160) will have some resiliency, after positioner (114) has been inserted through the opening (169a,169b) into sleeve (116a, 116b), opening (169a, 169b) will tend to return to its normal, unstretched state shown in FIG. 9. In this manner, and since the proximal end (126) of positioner (114) is located below opening (169a, 169b), in the depicted embodiment positioner (114) may only be removed from the sleeve (116a, 116b) by first pulling downwardly on the lower edge of opening (169a, 169b) and lower end portion (182) of inner panel (170) to expose the proximal end (115) of positioner (114). Such an arrangement will also tend to prevent positioner (114) from inadvertently being displaced from sleeve (116a, 116b). In addition, since openings (169a, 169b) of the depicted example will be located at or adjacent the hips of an infant, should positioner (114) become partially displaced from sleeve (116a, 116b) it is less likely that the displaced portion will present a hazard to the infant (e.g., the exposed portion of positioner (114) will extend away from the infant's head and neck). Of course various types of closures may be added to openings (169a, 169b) in order to further prevent inadvertent displacement of positioner (114). Such closures could include, for example, snap fasteners, buttons, hook and loop fasteners, zippers, and other means known to those skilled in the art.

Finally, FIG. 15 depicts yet another alternative embodiment of a garment system (210) comprising a garment (212) and a body positioner (214) located within an internal sleeve in garment (212). Garment system (210) and the components thereof are configured similar to that shown in FIGS. 1-7. However, an additional internal sleeve (290a, 290b) is provided on either side of garment (212), below the armpit region. A second body positioner (not shown) may be inserted into internal sleeve (290a, 290b), such as through opening (292b). The positioner may be configured similar to that previously described herein, albeit generally smaller in length and width. The additional body positioner (e.g., a wedge-shaped member) may be inserted in internal sleeve (290a, 290b) on the opposite side where positioner (214) is inserted. In this fashion, the additional positioner within second internal sleeve (290a, 290b) in order to prevent the infant from rolling onto their stomach.

It will be understood that the examples shown and described herein are merely exemplary, and may be modified by those skilled in the art. For example, the materials used in forming the various panels and other components of garment (12, 112) may comprise any of a variety of materials suitable for making garments. In one example, the front (40, 140), back (60, 160) and inner (70, 170) panels comprise synthetic or natural materials, such as various types of fabric (e.g., woven, nonwoven, and/or knitted). In addition, more than one material may be used in the garment (12, 112). For example, inner panel (70, 170) may be made from a material which is more elastic (capable of being stretched further, without permanent elongation) than the material used for front panel (40, 140) and/or back panel (60, 160). In this manner, inner panel (70, 170) may be configured to more securely retain positioner (14, 114) within the sleeve (16, 116). Similarly, the additional material used to provide retention pouches (80, 180) may be the same or different as that used for the various fabric panels. Any desirable pattern may be applied to these panels in order to provide an aesthetically pleasing appearance to the garment (12, 112). The fabric may be fire retardant, or otherwise treated so as to be fire retardant. In some embodiments, the various panels and retention pouches (80, 180) are made from cotton fabric.

In addition, while embodiments described herein use conventional stitching to attach or affix the various panels and other components to one another, any of a variety of other structures, features and techniques may be used in place of stitching. For example, one or more suitable adhesives or other types of fastening systems may be used for one or more of the attachments. In such alternatives, the stitch lines described herein are simply lines of attachment (e.g., inner panel (70, 170) may be glued to back panel (60, 160) along center attachment line, or simply centerline, (18, 118).

As yet another alternative, a single internal sleeve may be provided in the garment (12, 112), and formed similar to the sleeves (16, 116) described herein. In such a configuration, the single internal sleeve may be evenly positioned in the center of the back of the garment so as to extend across centerline (18, 118), equidistant therefrom on either side thereof.

As still another alternative, more than two internal sleeves may be provided for receiving body positioners. By way of example, internal sleeves may be provided on either or both sides of the garment (12, 112) extending downward from below the armpit (similarly to that shown in U.S. Patent Pub. No. 2007/0044199). Such side sleeves may be provided, in addition to, or in place of, sleeves (16, 116) described herein. In such an arrangement, positioners (bumpers) similar to positioners (14, 114) may be positioned in one or both of the side sleeves, and may be particularly useful in inhibiting an infant from rolling onto their stomach.

It will become readily apparent to those skilled in the art that examples described herein may have applicability beyond positioning an infant while sleeping so as to prevent positional plagiocephaly. For example, the garment systems described herein may be used for individuals of any age who have difficulty repositioning their body when in a supine position (e.g., people with spinal cord injuries). The garment systems described herein may be used with such individuals in order to, for example, prevent the occurrence of pressure sores (“bed sores”) due to laying in the same position over a long period of time. Various other ways in which the teachings herein may be used in other garments and/or other methods will be apparent to those of ordinary skill in the art.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I claim:

1. A garment system comprising:
(a) a torso-covering garment having at least one internal sleeve for receiving a body positioner therein; and
(b) a body positioner removably insertable in the at least one internal sleeve; wherein the garment further includes a retention member for releasably retaining the body positioner within the at least one internal sleeve, the garment system configured such that, when a person wearing the garment is in a supine position, the body positioner will position the person's upper torso in a predetermined orientation.

2. The garment system of claim 1, wherein the garment further comprises
front and back panels, each of which has inner and outer surfaces, the front and back panels attached to one another such that their respective inner surfaces are in a facing relationship; and
an inner panel attached to the inner surface of the back panel so as to form the at least one internal sleeve between the back and inner panels.

3. The garment system of claim 2, wherein the inner panel has an upper edge, and right and left side edges, the inner panel attached to the back panel along at least the upper and side edges of the inner panel, and further wherein the inner panel is attached to the back panel along a centerline extending from the upper edge of the inner panel, such that a pair of said internal sleeves are provided between the inner and back panels on either side of the centerline.

4. The garment system of claim 2, wherein the body positioner comprises an elongate wedge-shaped member.

5. The garment system of claim 3, wherein the body positioner comprises an elongate wedge-shaped member.

6. The garment system of claim 5, wherein the garment further comprises a pair of openings through which the body positioner is removably insertable into one of the internal sleeves, each opening associated with one of the internal sleeves.

7. The garment system of claim 6, wherein the back panel has a lower edge, said inner panel having a lower portion which extends below the lower edge of the back panel, and further wherein said openings are provided between the lower edge of the back panel and the inner panel.

8. The garment system of claim 6, wherein said openings are provided in the back panel, each opening located adjacent one of the internal sleeves.

9. The garment system of claim 6, wherein the garment further comprises a pair of retention members for retaining the body positioner within the internal sleeves, each retention member associated with one of the internal sleeves.

10. The garment system of claim 9, wherein said elongate wedge-shaped member has distal and proximal ends, and said retention members comprise a retention pouch located at one of end of each of the internal sleeves, said retention pouches configured to receive the proximal end of the wedge-shaped member inserted into the corresponding internal sleeve.

11. The garment system of claim 10, wherein the garment is configured to be worn by an infant such that, when the garment is worn by an infant, the internal sleeves extend from approximately the infant's hips to just below the neck along either side of the spine.

12. The garment system of claim 11, wherein said retention pouches have tapered widths corresponding to the cross-sectional shape of the proximal end of the wedge-shaped member such that said proximal end is snugly received in the retention pouch.

13. A garment system for preventing positional plagiocephaly in an infant, comprising:
(a) a torso-covering garment having a pair of internal sleeves for receiving a wedge-shaped bumper therein, wherein the garment includes
a front panel having inner and outer surfaces
a back panel having inner and outer surfaces, the back panel attached to the front panel such that the inner surface of the back panel is in facing relationship with the inner surface of the front panel
an inner panel having upper and side edges, the inner panel attached to the back panel along the upper and side edges of the inner panel, as well as along a centerline extending from the upper edge of the inner panel down the center of the inner panel such that said internal sleeves are provided between the inner and back panels on either side of the centerline; and
(b) a resilient wedge-shaped member removably insertable in either of said internal sleeves;
wherein the garment system is configured such that, when an infant wearing the garment is in a supine position, the resilient wedge-shaped member will elevate one side of the infant's back causing the infant's upper torso to be tilted with respect to the surface on which the infant lies.

14. The garment system of claim 13, wherein the garment further includes a pair of retention members for retaining the wedge-shaped member within the internal sleeves.

15. The garment system of claim 14, wherein each of said retention members comprises a retention pouch located at one of end of each of the internal sleeves, said retention pouches configured to receive the proximal end of the wedge-shaped member inserted into the corresponding internal sleeve.

16. The garment system of claim 15, wherein said retention pouches are provided by at least one fabric panel attached to the inner panel so as to form said retention pouches.

17. The garment system of claim 13, wherein said front and inner panels include upper and lower portions, with the lower portions of the front and inner panels configured to be releasably attached to one another such that said lower portions extend over the crotch area of an infant wearing the garment.

18. The garment system of claim 17, wherein the back panel has a lower edge, said lower portion of the inner panel extending below the lower edge of the back panel, and further wherein openings are provided between the lower edge of the back panel and the inner panel through which said resilient wedge-shaped member may be inserted into one of said internal sleeves.

19. The garment system of claim 18, wherein said front, back and inner panels comprise cotton fabric, and said resilient wedge-shaped member comprises a polymeric foam.

20. A torso-covering garment configured for receiving an elongate member therein, comprising:
(a) a front panel having inner and outer surfaces;
(b) a back panel having inner and outer surfaces, the back panel attached to the front panel such that the inner surface of the back panel is in facing relationship with the inner surface of the front panel;
(c) an inner panel having upper and side edges, the inner panel attached to the back panel along the upper and side edges of the inner panel, as well as along a centerline extending from the upper edge of the inner panel down the center of the inner panel such that said a pair of internal sleeves are provided between the inner and back panels with the sleeves located on opposite sides of the centerline;
(d) a pair of retention pouches, one of said pouches located at the lower end of each of the internal sleeves.
